(19) United States
(12) Patent Application Publication
(10) Pub. No.: US 2025/0093347 A1
(43) Pub. Date: Mar. 20, 2025

(54) BIOSENSOR AND METHOD FOR MANUFACTURING SAME, AND BIOCHIP

(71) Applicants: Beijing BOE Technology Development Co., Ltd., Beijing (CN); BOE Technology Group Co., Ltd., Beijing (CN)

(72) Inventors: Ruijun DENG, Beijing (CN); Shicai WANG, Beijing (CN); Yang SONG, Beijing (CN); Ding DING, Beijing (CN)

(21) Appl. No.: 18/578,665

(22) PCT Filed: Jan. 17, 2023
(86) PCT No.: PCT/CN2023/072711

§ 371 Date: Jan. 11, 2024

(51) Int. Cl. G01N 33/543; B01L 3/00

(52) U.S. Cl. CPC G01N 33/5438; B01L 3/502761; B01L 2200/0647; B01L 2200/12; B01L 2300/0645; B01L 2300/0819; B01L 2300/0883

(57) ABSTRACT

Provided is a biosensor. The biosensor includes: a base substrate; a plurality of detection units disposed on a side of the base substrate, wherein each of the plurality of detection units includes a gate; a first drive signal line disposed on the side of the base substrate and electrically connected to the gate; and a cover plate fixedly connected to the side of the base substrate, wherein a plurality of recesses are defined in a side, proximal to the base substrate, of the cover plate.

### CROSS-REFERENCE TO RELATED APPLICATION

This application is a U.S. national stage of international application No. PCT/CN2023/072711, filed on Jan. 17, 2023, the disclosure of which is incorporated herein by reference in its entirety.

### TECHNICAL FIELD

The present disclosure relates to the field of display technologies, and in particular, relates to a biosensor and a method for manufacturing the same, and a biochip.

### BACKGROUND

Biosensors are used in various biochips, and are capable of detecting concentrations of biomolecules in a liquid under detection (for example, body fluids, bloods, and the like in human bodies).

### SUMMARY

Embodiments of the present disclosure provide a biosensor and a method for manufacturing the same, and a biochip. The technical solutions are as follows.

In some embodiments of the present disclosure, a biosensor is provided. The biosensor includes:
a base substrate;
a plurality of detection units disposed on a side of the base substrate, wherein each of the plurality of detection units includes a gate;
a first drive signal line disposed on the side of the base substrate and electrically connected to the gate; and
a cover plate fixedly connected to the side of the base substrate, wherein a plurality of recesses are defined in a side, proximal to the base substrate, of the cover plate.

In some embodiments, each of the plurality of detection units further includes a graphene layer and a probe modification layer on a side, facing away from the base substrate, of the graphene layer, wherein an orthogonal projection of the graphene layer on the base substrate is not overlapped with an orthogonal projection of the gate on the base substrate, an orthogonal projection of the probe modification layer on the base substrate is within the orthogonal projection of the graphene layer on the base substrate, and the probe modification layer contains an antibody protein for reacting with a specific antigenic protein in a liquid under detection.

In some embodiments, the probe modification layers in the plurality of detection units contain different types of antibody proteins.

In some embodiments, each of the plurality of detection units further includes a source and a drain that are lapped with the graphene layer, wherein the orthogonal projection of the probe modification layer on the base substrate is between an orthogonal projection of the source on the base substrate and an orthogonal projection of the drain on the base substrate.

In some embodiments, the gate, the source, and the drain are disposed on a same layer and made of a same material.

In some embodiments, each of the plurality of detection units further includes an insulative layer on a side, facing away from the base substrate, of the source and the drain, wherein the insulative layer is configured to wrap the source and the drain, wherein an orthogonal projection of the insulative layer on the base substrate is not overlapped with the orthogonal projection of the gate on the base substrate and the orthogonal projection of the probe modification layer on the base substrate.

In some embodiments, the biosensor further includes: a second drive signal line and a third drive signal line that are disposed on the side of the base substrate, wherein the second drive signal line is electrically connected to the source, the third drive signal line is electrically connected to the drain,

none of an end portion, facing away from the gate, of the first drive signal line, an end portion, facing away from the source, of the second drive signal line, and an end portion, facing away from the drain, of the third drive signal line is covered by the cover plate, and the insulative layer is further configured to wrap portions of the second drive signal line and the third drive signal line in the plurality of recesses.

In some embodiments, a liquid inlet and a plurality of liquid outlets are defined in a side, facing away from the base substrate, of the cover plate, wherein the liquid inlet is connected to the plurality of recesses, the plurality of liquid outlets are in one-to-one correspondence to the plurality of recesses and are connected to corresponding recesses; and

orthogonal projections of the plurality of recesses on the base substrate are not overlapped with an orthogonal projection of the liquid inlet on the base substrate and orthogonal projections of the plurality of liquid outlets on the base substrate.

In some embodiments, the cover plate further includes a plurality of first microfluidic channels in one-to-one correspondence to the plurality of recesses, wherein first ends of the plurality of first microfluidic channels are connected to the liquid inlet, and second ends of the plurality of first microfluidic channels are connected to corresponding recesses.

In some embodiments, each of the plurality of first microfluidic channels includes a plurality of lengthening sub-channels and a plurality of connecting sub-channels, wherein the plurality of lengthening sub-channels and the plurality of connecting sub-channels are alternately arranged and are sequentially connected.

In some embodiments, the cover plate further includes a plurality of second microfluidic channels in one-to-one correspondence to the plurality of recesses, wherein first ends of the plurality of second microfluidic channels are connected to corresponding recesses, and second ends of the plurality of second microfluidic channels are connected to corresponding liquid outlets.

In some embodiments of the present disclosure, a biochip is provided. The biochip includes: a drive assembly and a biosensor electrically connected to the drive assembly, wherein the biosensor is the above biosensor.

In some embodiments of the present disclosure, a method for manufacturing a biosensor is provided. The method includes:
forming a plurality of detection units on a side of a base substrate, wherein each of the plurality of detection units includes a gate;
forming a first drive signal line electrically connected to the gate on the side of the base substrate; and
securing a cover plate on the side of the base substrate, wherein a plurality of recesses configured to accommodate the plurality of detection units are defined in a side, proximal to the base substrate, of the cover plate.

In some embodiments, forming the plurality of detection units on the side of the base substrate includes:
forming a graphene layer, a gate, a source, and a drain on the side of the base substrate, wherein the source and the drain are both lapped with the graphene layer, and an orthogonal projection of the gate on the base substrate is not overlapped with an orthogonal projection of the graphene layer on the base substrate; and
forming a probe modification layer on a side, facing away from the base substrate, of the graphene layer upon securing the cover plate on the side of the base substrate,
wherein an orthogonal projection of the probe modification layer on the base substrate is within the orthogonal projection of the graphene layer on the base substrate and between an orthogonal projection of the source on the base substrate and an orthogonal projection of the drain on the base substrate, and the probe modification layer contains an antibody protein for reacting with a specific antigenic protein in a liquid under detection.

In some embodiments, a liquid inlet and a plurality of liquid outlets are defined in a side, facing away from the base substrate, of the cover plate, wherein the liquid inlet is connected to the plurality of recesses, and the plurality of liquid outlets are in one-to-one correspondence to the plurality of recesses and are connected to corresponding recesses; and forming the probe modification layer on the side, facing away from the base substrate, of the graphene layer includes:
forming a binding substance on the graphene layer by injecting a binding solution to the plurality of recesses through the liquid inlet; and
sequentially injecting a solution containing different types of antibody proteins to the plurality of recesses through the plurality of liquid outlets, such that the antibody proteins are bound with the binding substance.

### BRIEF DESCRIPTION OF DRAWINGS

For clearer description of the technical solutions in the embodiments of the present disclosure, the accompanying drawings required for describing the embodiments are briefly introduced. Apparently, the accompanying drawings in the following description show merely some embodiments of the present disclosure, and those of ordinary skill in the art may still derive other drawings from these accompanying drawings without any creative efforts.

FIG. 1 is a top view of a biosensor according to some embodiments of the present disclosure;

FIG. 2 is a top view of a detecting back plate in the biosensor shown in FIG. 1;

FIG. 3 is a schematic diagram of a film structure of the detecting back plate shown in FIG. 2 at a position of A-A′;

FIG. 4 is a schematic structural diagram of a cover plate in the biosensor shown in FIG. 1;

FIG. 5 is a schematic diagram of a film structure of the biosensor shown in FIG. 1 at a position of B-B′;

FIG. 6 is a top view of another biosensor according to some embodiments of the present disclosure;

FIG. 7 is a schematic diagram of a cover plate on another side of the biosensor shown in FIG. 6;

FIG. 8 is a locally enlarged diagram of the biosensor shown in FIG. 6 at a position of C;

FIG. 9 is a schematic diagram of a film structure of the biosensor shown in FIG. 6 at a position of D-D′;

FIG. 10 is a schematic diagram of a film structure of the biosensor shown in FIG. 6 at a position of E-E′;

FIG. 11 is a top view of a graphene layer, a source, and a drain according to some embodiments of the present disclosure;

FIG. 12 is a top view of a detection unit according to some embodiments of the present disclosure;

FIG. 13 is a schematic diagram of a relationship of current change rates at different concentrations of Aβ40 according to some embodiments of the present disclosure;

FIG. 14 is a detecting curve of detecting Aβ40 by a detection unit according to some embodiments of the present disclosure;

FIG. 15 is a flowchart of a method for manufacturing a biosensor according to some embodiments of the present disclosure; and

FIG. 16 is a flowchart of another method for manufacturing a biosensor according to some embodiments of the present disclosure.

### DETAILED DESCRIPTION

To make the objectives, technical solutions, and advantages of the present disclosure clearer, the embodiments of the present disclosure are further described in detail hereinafter with reference to the accompanying drawings.

The biosensor includes cavities for bearing the liquid under detection, and detection units are disposed in the cavities. Currently, in the biosensor, external metal wires are determined as gate signal applying electrodes in the detection units. In detecting concentrations of the biomolecules in the liquids under detection in the cavities by the detection units, the metal wires are inserted in the cavities to apply gate signals to the liquids under detection in the cavities, such that the concentrations of the biomolecules in the liquids under detection are detected by the biosensor.

However, in the current process of detecting the liquid under detection by the biosensor, the liquid is prone to volatilization, and thus an accuracy of detecting the concentrations of the biomolecules in the liquid for detection is poor.

A cover plate capable of covering a cavity of the biosensor is disposed in the biosensor to reduce volatility of a liquid under detection in detecting the liquid under detection by the biosensor. As external metal wires require to insert to the cavity of the biosensor, holes connected to the cavity of the biosensor are required in the cover plate, such that the metal wires are capable of inserting to the cavity of the biosensor over the holes.

However, for convenient and quick insertion of the metal wires to the cavity of the biosensor, the metal wires require to be clearance fitted with the holes in the cover plate. As such, after the metal wires are inserted to the holes, a gap is present between the metal wires and the holes, such that the sealing effect of the cover plate to the cavity of the biosensor is poor, and the liquid under detection in the cavity may be volatilized.

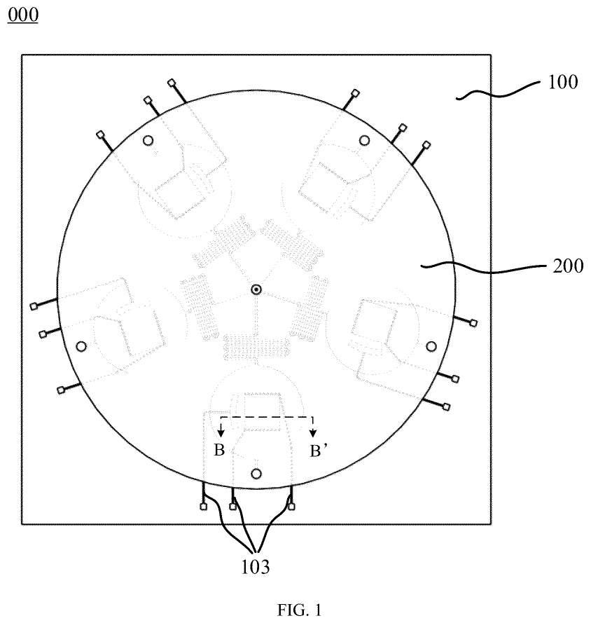

Referring to FIG. 1, FIG. 1 is a top view of a biosensor according to some embodiments of the present disclosure. The biosensor 000 includes a detecting back plate 100 and a cover plate 200.

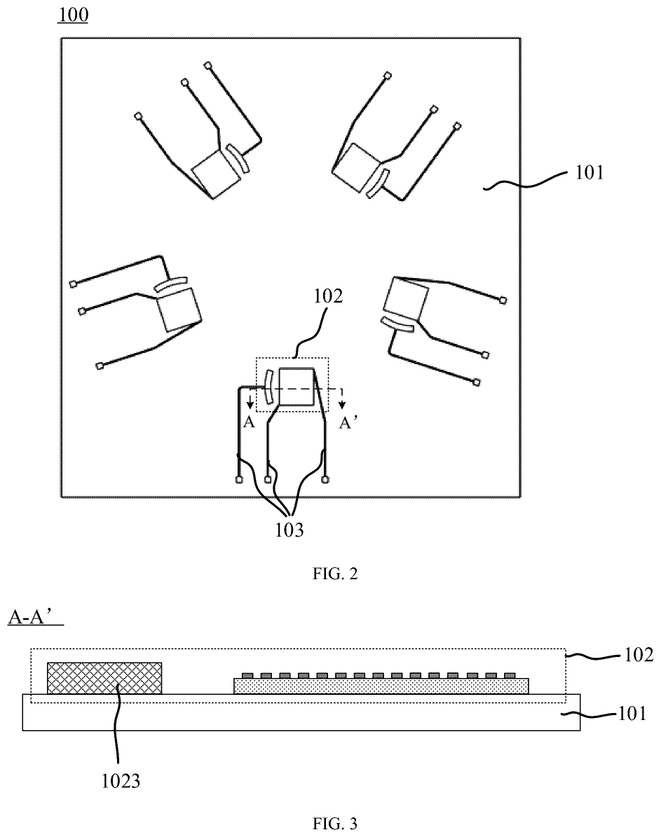

For clearer understanding of the structure of the detecting back plate 100 in the biosensor 000, FIG. 2 and FIG. 3 are referred. FIG. 2 is a top view of a detecting back plate in the biosensor shown in FIG. 1. FIG. 3 is a schematic diagram of a film structure of the detecting back plate shown in FIG. 2 at a position of A-A′. The detecting back plate 100 in the biosensor 000 includes a base substrate 101 and a plurality of detection units 102 on a side of the base substrate. Each of the plurality of detection units 102 in the detecting back plate 100 is configured to detect concentrations of biomolecules in a liquid under detection. Illustratively, the biomolecules in the liquid under detection are various antigenic proteins in the liquid under detection.

The detecting back plate 100 further includes a drive signal line 103 on the side of the base substrate 101 and electrically connected to the detection units 102. The drive signal line 103 is configured to apply a drive signal to the detection units 102, such that the detection units 102 operate normally.

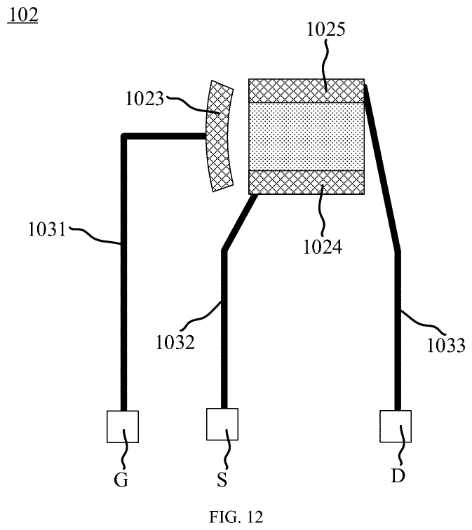

In the present disclosure, each of the plurality of detection units 102 in the detecting back plate 100 includes a gate 1023, and the drive signal line 103 electrically connected to the detection units 102 includes a first drive signal line electrically connected to the gate 1023 (not shown in the drawings but shown in FIG. 12).

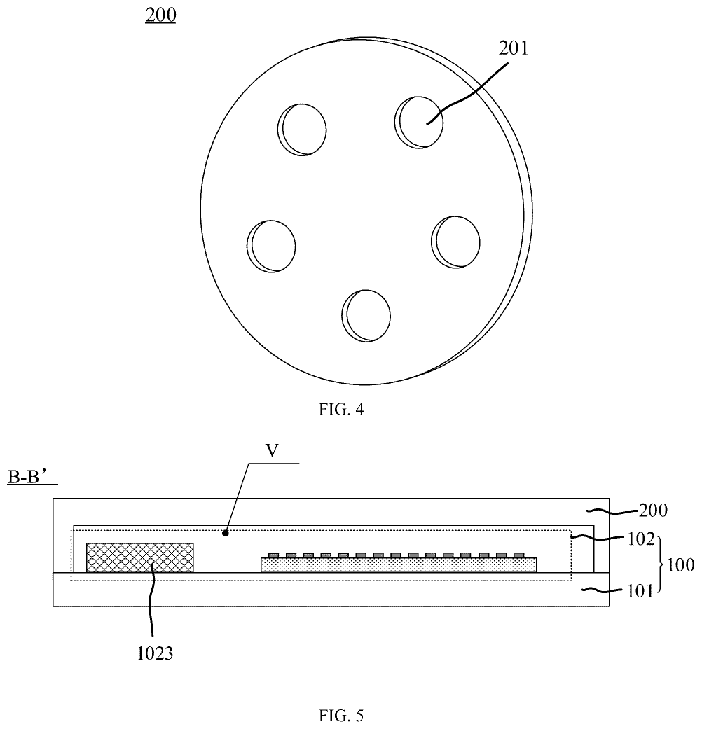

The cover plate 200 in the biosensor 000 is fixedly connected to the side of the base substrate 101 in the detecting back plate 100. For clearer understanding of the structure of the cover plate 200 in the biosensor 000, FIG. 1 and FIG. 4 are referred. FIG. 4 is a schematic structural diagram of a cover plate in the biosensor shown in FIG. 1. A plurality of recesses 201 are defined in a side, proximal to the base substrate 101, of the cover plate 200. The plurality of recesses 201 in the cover plate 200 are configured to accommodate the plurality of detection units 102 in the detecting back plate 100. In some embodiments, the plurality of recesses 201 in the cover plate 200 are in one-to-one correspondence to the plurality of detection units 102, and each recess 201 is configured to accommodate the corresponding detection unit 102. In some embodiments, each recess 201 in the cover plate 200 is configured to accommodate two or more detection units 102, which is not limited in the embodiments of the present disclosure. It should be noted that the following embodiments are described by taking one recess 201 being configured to accommodate one detection unit 102 as an example for convenient description.

In the embodiments of the present disclosure, as shown in FIG. 5, FIG. 5 is a schematic diagram of a film structure of the biosensor shown in FIG. 1 at a position of B-B′. After the cover plate 200 is secured on the base substrate 101 in the detecting back plate 100, each recess 201 in the cover plate 200 and the detecting back plate 100 form a detection cavity V, and the detection unit 102 is disposed in the detection cavity V. As such, after a liquid under detection is accommodated in the detection cavity V, and a drive signal is applied to the detection unit 102 through a drive signal line 103, concentrations of biomolecules in the liquid under detection are detected by the detection unit 102. As the first drive signal line electrically connected to the gate 1023 in the detection unit 102 is integrated in the detecting back plate 100, the detecting back plate 100 directly applies a gate signal to the detection unit 102 through the first drive signal line, without the need to holes in the cover plate 200 for inserting metal wires to the detection cavity V, such that the sealing between the cover plate 200 and the detecting back plate 100 is great, volatility of the liquid under detection in the detection cavity V is efficiently reduced, and an accuracy of detecting the concentrations of the biomolecules in the liquid under detection by the detection unit 102 is great.

In summary, the biosensor in the embodiments of the present disclosure includes a detecting back plate and a cover plate. The detecting back plate includes a base substrate, a plurality of detection units on a side of the base substrate, and a first drive signal line electrically connected to a gate in the detection unit. After the cover plate is secured on the base substrate in the detecting back plate, each recess in the cover plate and the detecting back plate form a detection cavity, and the detection unit is in the detection cavity. As such, after a liquid under detection is accommodated in the detection cavity, and a drive signal is applied to the detection unit through a drive signal line, the concentrations of biomolecules in the liquid under detection are detected by the detection unit. As the first drive signal line electrically connected to the gate in the detection unit is integrated in the detecting back plate, the detecting back plate directly applies a gate signal to the detection unit through the first drive signal line, without the need to holes in the cover plate for inserting metal wires to the detection cavity, such that the sealing between the cover plate and the detecting back plate is great, volatility of the liquid under detection in the detection cavity is efficiently reduced, and an accuracy of detecting the concentrations of the biomolecules in the liquid under detection by the detection unit is great.

In some embodiments, the cover plate 200 in the biosensor 000 is made of polydimethylsiloxane (PDMS). As such, plasma bombardment is performed on a side of the cover plate 200 disposed with the recess 201 before the cover plate 200 is covered on the detecting back plate 100, such that the cover plate 200 is closely attached to the detecting back plate 100 upon being covered on the detecting back plate 100.

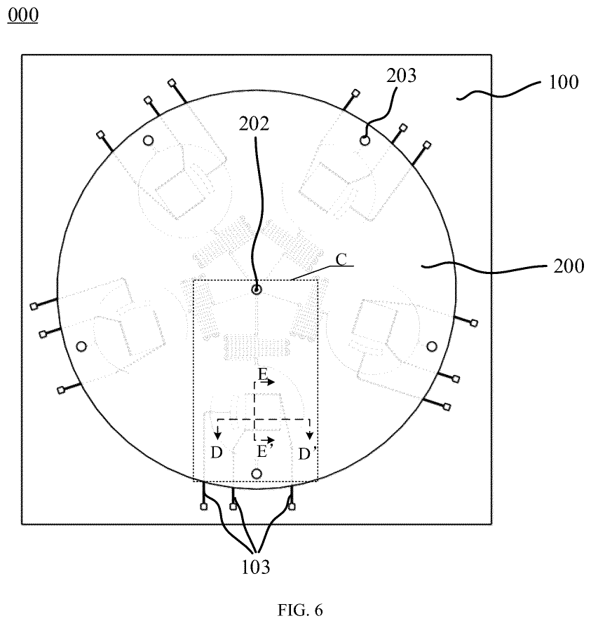

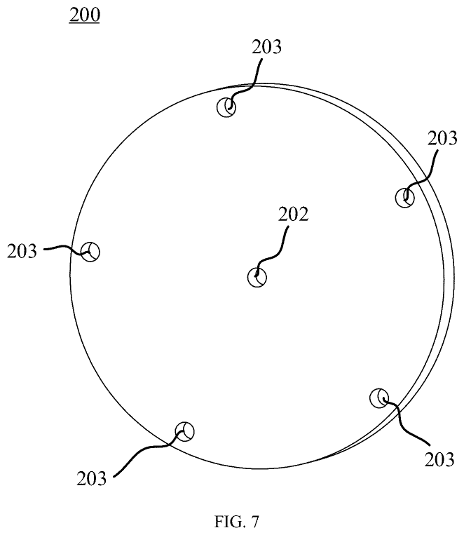

In the embodiments of the present disclosure, as shown in FIG. 6 and FIG. 7, FIG. 6 is a top view of another biosensor according to some embodiments of the present disclosure. FIG. 7 is a schematic diagram of a cover plate on another side of the biosensor shown in FIG. 6. A liquid inlet 202 and a plurality of liquid outlets 203 are defined in a side, facing away from the base substrate 101 in the detecting back plate 100, of the cover plate 200 in the biosensor 000. The liquid inlet 202 in the cover plate 200 is connected to the plurality of recesses 201, the plurality of liquid outlets 203 in the cover plate 200 are in one-to-one correspondence to the plurality of recesses 201 and are connected to the corresponding recesses 201.

In the present disclosure, in the case that the liquid under detection requires to be detected by the biosensor, the liquid under detection is injected to the liquid inlets 202 in the cover plate 200, and the liquid under detection flows in directions of the recesses 201 and flows in the detection cavities V, such that the detection unit 102 in each detection cavity V detects the concentrations of the biomolecules in the liquid under detection. Upon detection on the liquid under detection, the liquids under detection in the detection cavities V are discharged through the liquid outlets 203 in the cover plate 200.

Orthogonal projections of the plurality of recesses 201 in the cover plate 200 on the base substrate 101 are not overlapped with an orthogonal projection of the liquid inlet 202 on the base substrate 101 and orthogonal projections of the plurality of liquid outlets 203 on the base substrate 101. In this case, a probability of volatilization of the liquid under detection in the detection cavity V through the liquid inlet 202 and the liquid outlets 203 is low, such that the sealing of the detection cavity V is improved on the premise that the liquid under detection is normally injected to the detection cavity V, and the volatility of the liquid under detection in the detection cavity V is further reduced.

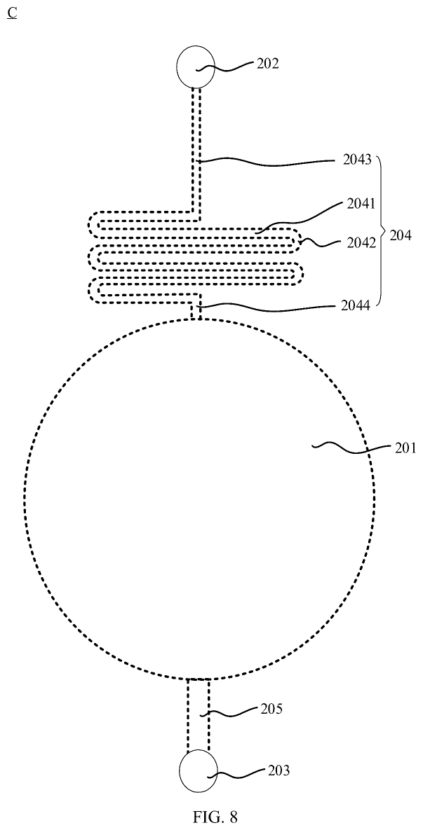

In some embodiments, as shown in FIG. 8, FIG. 8 is a locally enlarged diagram of the biosensor shown in FIG. 6 at a position of C. The cover plate 200 in the biosensor 000 further includes a plurality of first microfluidic channels 204. The plurality of first microfluidic channels 204 are in one-to-one correspondence to the plurality of recesses 201. First ends of the plurality of first microfluidic channels 204 are connected to the liquid inlet 202, and second ends of the plurality of first microfluidic channels 204 are connected to the corresponding recesses 201. As such, upon injection of the liquid under detection to the liquid inlet 202 in the cover plate 200, the liquid under detection flows in the corresponding recesses 201 through the first microfluidic channels 204, such that the liquid under detection flows in the detecting cavities V.

In the embodiments of the present disclosure, each of the plurality of first microfluidic channels 204 includes a plurality of lengthening sub-channels 2041 and a plurality of connecting sub-channels 2042. The plurality of lengthening sub-channels 2041 and the plurality of connecting sub-channels 2042 are alternately arranged in each first microfluidic channel 204. For example, one connecting sub-channel 2042 is disposed between two adjacent lengthening sub-channels 2041. The plurality of lengthening sub-channels 2041 and the plurality of connecting sub-channels 2042 are connected sequentially in each first microfluidic channel 204.

Illustratively, for any three adjacent lengthening sub-channels 2041, a first end of a first lengthening sub-channel is connected to a first end of a second lengthening sub-channel through one connecting sub-channel 2042, and a second end of the second lengthening sub-channel is connected to a second end of a third lengthening sub-channel through another connecting sub-channel 2042. The first end of the lengthening sub-channel refers to an end of the lengthening sub-channel on a left side shown in FIG. 8, and the second end of the lengthening sub-channel refers to an end of the lengthening sub-channel on a right side shown in FIG. 8. The first lengthening sub-channel is a lengthening sub-channel in the middle of the three lengthening sub-channels 2041, the second lengthening sub-channel is a lengthening sub-channel of the three lengthening sub-channels 2041 on one side of the first lengthening sub-channel, and the third lengthening sub-channel is a lengthening sub-channel of the three lengthening sub-channels 2041 on the other side of the first lengthening sub-channel.

It should be noted that extension directions of the lengthening sub-channels 2041 in each first microfluidic channel 204 are straight and are parallel to each other. That is, a plurality of lengthening sub-channels 2041 in each first microfluidic channel 204 are arranged in parallel. An extension direction of the connecting sub-channel 2042 for connecting two adjacent lengthening sub-channels 2041 in each first microfluidic channel 204 is annular, and an orientation of the connecting sub-channel 2042 connected to the first end of each lengthening sub-channel 2041 is opposite to an orientation of the connecting sub-channel 2042 connected to the second end of each lengthening sub-channel 2041.

In this case, an entire extension length of the first microfluidic channel 204 is great by disposing a plurality of parallel lengthening sub-channels 2041 in the first microfluidic channel 204. As such, the liquid under detection is fully mixed in flowing in the first microfluidic channel 204, and the liquid under detection in the detection cavity V is not prone to volatilization outward through the liquid inlet 202, such that the sealing of the detection cavity V is further improved.

In some embodiments, the lengthening sub-channels 2041 in each first microfluidic channel 204 are disposed between the liquid inlet 202 and the corresponding recess 201. Each first microfluidic channel 204 further includes a first connecting sub-channel 2043 and a second connecting sub-channel 2044. A first end of the first connecting sub-channel 2043 is connected to the liquid inlet 202, and a second end of the first connecting sub-channel 2043 is connected to a second end of the lengthening sub-channel 2041 in the plurality of lengthening sub-channels 2041 most proximal to the liquid inlet 202. A first end of the second connecting sub-channel 2044 is connected to the corresponding recess 201, and a second end of the second connecting sub-channel 2044 is connected to a second end of the lengthening sub-channel 2041 in the plurality of lengthening sub-channels 2041 most proximal to the recess 201. In each first microfluidic channel 204, an extension direction of the first connecting sub-channel 2043 is parallel to an extension direction of the second connecting sub-channel 2044 and is perpendicular to an extension direction of the lengthening sub-channel 2041.

In the present disclosure, the liquid inlet 202 in the cover plate 200 is disposed in the center of the cover plate 200. That is, an axis line of the liquid inlet 202 is coincided with an axis line of the cover plate 200. The plurality of recesses 201 in the cover plate 200 are uniformly disposed around the liquid inlet 202. That is, distances between the recesses 201 and the liquid inlet 202 are the same. In this case, entire extension lengths of the first microfluidic channels 204 in the cover plate 200 are equal, such that volumes of the liquids under detection flowed in the detecting cavities V are equal in the case that the liquids under detection injected from the liquid inlet 202 flow to the detecting cavities V through the plurality of first microfluidic channels 204, and the detection units 102 in the detecting cavities V accurately detect the liquids under detection.

In some embodiments, as shown in FIG. 8, the cover plate 200 in the biosensor 000 further includes a plurality of second microfluidic channels 205. The plurality of second microfluidic channels 205 are in one-to-one correspondence to the plurality of recesses 201 and the plurality of liquid outlets 203. First ends of the plurality of second microfluidic channels 205 are connected to the corresponding recesses 201, and second ends of the plurality of second microfluidic channels 205 are connected to corresponding liquid outlets 203. As such, upon detection on the liquids under detection by the detection units 102 in the detecting cavities V, the liquids under detection are discharged from the second microfluidic channels 205 and the corresponding liquid outlets 203, such that the liquids under detection in the detecting cavities V are discharged.

Illustratively, in the cover plate 200, each liquid outlet 203 is disposed in a side, facing away from the liquid outlet 203, of the corresponding recess 201, such that a distance between the liquid outlet 203 and the liquid inlet 202 is great, and the interference between the liquid entering process from the liquid inlet 202 and the liquid discharging process from the liquid outlet 203 is avoided.

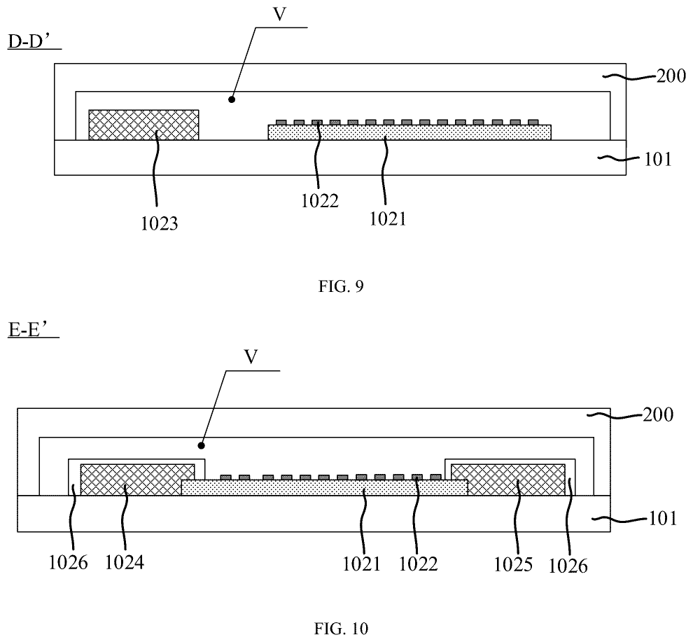

In the embodiments of the present disclosure, as shown in FIG. 9 and FIG. 10, FIG. 9 is a schematic diagram of a film structure of the biosensor shown in FIG. 6 at a position of D-D′. FIG. 10 is a schematic diagram of a film structure of the biosensor shown in FIG. 6 at a position of E-E′. Each detection unit 102 in the detecting back plate 100 in the biosensor 000 further includes a graphene layer 1021 and a probe modification layer 1022 on a side, facing away from the base substrate 101, of the graphene layer 1021. An orthogonal projection of the probe modification layer 1022 on the base substrate 101 is within the orthogonal projection of the graphene layer 1021 on the base substrate 101, and the probe modification layer 1022 contains an antibody protein for reacting with a specific antigenic protein in a liquid under detection.

In the case that the liquid under detection is accommodated in the detection cavity V in the biosensor 000, the detection unit 102 in the detection cavity V detects a concentration of a specific antigenic protein in the liquid under detection. Illustratively, an antigen-antibody immune reaction occurs between the antibody protein in the probe modification layer 1022 in the detection unit 102 and the antigenic protein, and a current in the graphene layer 1021 changes upon the antigen-antibody immune reaction. In the case that concentrations of the antigenic proteins in the liquid under detection are different, reaction degrees between the antigenic proteins and the antibody proteins in the probe modification layer 1022 are different, such that currents in the graphene layer 1021 change differentially. As such, the concentration of the specific antigenic protein in the liquid under detection is determined by detecting the change of the current in the graphene layer 1021 in the detection unit 102.

In the present disclosure, the probe modification layers 1022 in the plurality of detection units 102 in the detecting back plate 100 contain different types of antibody proteins. For example, the probe modification layer 1022 in each detection unit 102 in the detecting back plate 100 contains one type of antibody protein, such that the plurality of detection units 102 respectively detect the concentrations of the different types of antigenic proteins in the liquid under detection. Illustratively, upon injection of the liquids under detection to the detecting cavities V by the liquid inlet 202, the antibody protein in the probe modification layer 1022 in the detection unit 102 in the detection cavity Vis reacted with one antigenic protein in the liquid under detection in the detection cavity V, such that the concentration of the antigenic protein is detected. As such, by disposing a plurality of detecting cavities V in the biosensor 000 and disposing the detection unit 102 in each detection cavity V, the biosensor 000 is capable of simultaneously detecting concentrations of various antigenic proteins in the liquid under detection, and thus the efficiency of detecting the liquid under detection is efficiently improved.

Illustratively, assuming that the various antigenic proteins in the liquid under detection are at least two of Aβ40, Aβ42, GFAP, NfL, p-tau 181, and p-tau 217, the antibody proteins corresponding to the antigenic proteins are used, and the probe modification layers 1022 in the detection units 102 are disposed in different detecting cavities V, such that the probe modification layer 1022 in each detection unit 102 contains the corresponding antibody protein.

In the embodiments of the present disclosure, as shown in FIG. 9 and FIG. 10, each detection unit 102 in the detecting back plate 100 in the biosensor 000 further includes a source 1024 and a drain 1025 that are lapped with the graphene layer 1021. In each detection unit 102, an orthogonal projection of the gate 1023 on the base substrate 101 is not overlapped with an orthogonal projection of the graphene layer 1021 on the base substrate 101, and the orthogonal projection of the probe modification layer 1022 on the base substrate 101 is between an orthogonal projection of the source 1024 on the base substrate 101 and an orthogonal projection of the drain 1025 on the base substrate 101.

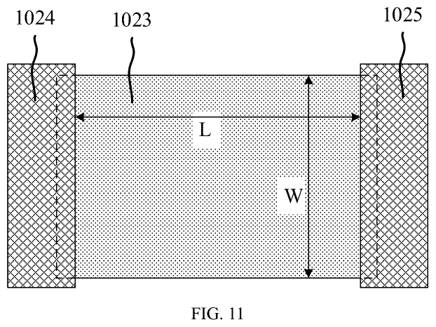

For clearer understanding of the relationship of the graphene layer 1021, the source 1024, and the drain 1025 in the detection unit 102, FIG. 11 is referred. FIG. 11 is a top view of a graphene layer, a source, and a drain according to some embodiments of the present disclosure. In the detection unit 102, the source 1024 and the drain 1025 are respectively disposed on two sides of the graphene layer 1021, the orthogonal projection of the source 1024 on the base substrate 101 is overlapped with the orthogonal projection of the graphene layer 1021 on the base substrate 101, and the orthogonal projection of the drain 1025 on the base substrate 101 is overlapped with the orthogonal projection of the graphene layer 1021 on the base substrate 101, such that the source 1024 and the drain 1025 are respectively lapped with the two sides of the graphene layer 1021.

It should be noted that a distance between the source 1024 and the drain 1025 in the detection unit 102 requires to be greater than or equal to 20 μm, a width of an overlapped portion of the source 1024 and the graphene layer 1021 and a width of an overlapped portion of the drain 1025 and the graphene layer 1021 require to be greater than or equal to 2 μm, and a width of a protrusion portion of the source 1024 relative to the graphene layer 1021 and a width of a protrusion portion of the drain 1025 relative to the graphene layer 1021 require to be greater than or equal to 2 μm, such that the precise of manufacturing the source 1024 and the drain 1025 is met, and the effect of electric connection in lapping the source 1024 and the drain 1025 with the graphene layer 1021 is ensured.

It should be further noted that a region of the graphene layer 1021 between the source 1024 and the drain 1025 belongs to a channel region. A length L of the channel region refers to a distance between the source 1024 and the drain 1025, and a width W of the channel region refers to a width of the graphene layer 1021. The length L of the channel region in the graphene layer 1021 requires to be greater than or equal to 20 μm, and the width W of the channel region in the graphene layer 1021 requires to be greater than or equal to 10 μm, such that a proportion of the width to length W/L of the channel region in the graphene layer 1021 is greater than or equal to 1:2, and the electric performance of the detection unit 102 is great. In some embodiments, the length L and the width W of the channel region in the graphene layer 1021 are in a millimeter scale. For example, the length L and the width W are both 3 mm. As such, an area of the graphene layer 1021 in the detection unit 102 on the base substrate 101 is great, and the sensitively of the detection unit 102 is not affected on the premise that the detecting efficiency of the detection unit 102 is great.

In some embodiments, as in the detection unit 102, the orthogonal projection of the gate 1023 on the base substrate 101 is not overlapped with the orthogonal projection of the graphene layer 1021 on the base substrate 101, the gate 1023, the source 1024, and the drain 1025 are disposed on a same layer and made of a same material. That is, the gate 1023, the source 1024, and the drain 1025 are formed by the one patterning process. As such, in forming the gate 1023, the source 1024, and the drain 1025 by the one patterning process, the complexity of manufacturing the detection unit 102 is efficiently simplified. It should be noted that the one patterning processes herein and in the above embodiments include photoresist coating, exposing, developing, etching, and photoresist removing.

In the embodiments of the present disclosure, the detection unit 102 further includes an insulative layer 1026 on a side, facing away from the base substrate 101, of the source 1024 and the drain 1025. The insulative layer 1026 is configured to wrap the source 1024 and the drain 1025. That is, the insulative layer 1026 covers a face, facing away from the base substrate 101, of the source 1024 and a side face of the source 1024. Similarly, the insulative layer 1026 covers a face, facing away from the base substrate 101, of the drain 1025 and a side face of the drain 1025. In this case, by insulating the source 1024 from the drain 1025 by the insulative layer 1026, the liquid under detection in the detection cavity V does not conduct the source 1024 and the drain 1025, and only the graphene layer 1021 conduct the source 1024 and the drain 1025, such that the detection unit 102 normally detects the liquid under detection.

In the present disclosure, an orthogonal projection of the insulative layer 1026 in the detection unit 102 on the base substrate 101 is not overlapped with the orthogonal projection of the gate 1023 on the base substrate 101 and the orthogonal projection of the probe modification layer 1022 on the base substrate 101. In this case, in loading the gate signal on the gate 1023, carriers are generated in the channel region in the graphene layer 1021 under the conjunction action of the gate signal loaded on the gate 1023 and water molecules in the liquid under detection, such that the channel region in the graphene layer 1021 is conducted. As such, when an electric signal is loaded on the source 1024, the source 1024 transmits the electric signal to the drain 1025 through the channel region in the graphene layer 1021, such that a current is formed between the source 1024 and the drain 1025. As the orthogonal projection of the insulative layer 1026 in the detection unit 102 on the base substrate 101 is not overlapped with the orthogonal projection of the probe modification layer 1022 on the base substrate 101, the liquid under detection is in direct contact with the probe modification layer 1022, such that an antigen-antibody immune reaction occurs between the antibody protein in the probe modification layer 1022 and the specific antigenic protein in the liquid under detection. Charges are generated in the antigen-antibody immune reaction, and a number of the carriers generated in the channel region in the graphene layer 1021 changes under the effect of the charges, such that the current between the source 1024 and the drain 1025 changes. As in the case that concentrations of the antigenic proteins in the liquid under detection are different, reaction degrees between the antigenic proteins and the antibody proteins in the probe modification layer 1022 are different, currents between the source 1024 and the drain 1025 change differentially. As such, the concentrations of the corresponding antigenic protein in the liquid under detection are acquired by detecting the change degree of the current between the source 1024 and the drain 1025.

In some embodiments, as shown in FIG. 12, FIG. 12 is a top view of a detection unit according to some embodiments of the present disclosure. The drive signal line 103 includes a first drive signal line 1031 electrically connected to the gate 1023 in the detection unit 102, a second drive signal line 1032 electrically connected to the source 1024 in the detection unit 102, and a third drive signal line 1033 electrically connected to the drain 1025 in the detection unit 102. The first drive signal line 1031 and the gate 1023 are dispose on a same layer and made of a same material, the second drive signal line 1032 and the source 1024 are dispose on a same layer and made of a same material, and the third drive signal line 1033 and the drain 1025 are dispose on a same layer and made of a same material, such that the difficulty of manufacturing the detecting back plate 100 is further reduced.

In the present disclosure, the insulative layer 1026 in the detection unit 102 is further configured to wrap portions of the second drive signal line 1032 and the third drive signal line 1033 in the plurality of recesses 201 (that is, the detection cavity V), such that the second drive signal line 1032 and the third drive signal line 1033 in the detection cavity V are insulated from each other by the insulative layer to avoid conduction of the second drive signal line 1032 and the third drive signal line 1033 by the liquid under detection in the detection cavity V.

In some embodiments, none of an end portion, facing away from the gate 1023, of the first drive signal line 1031, an end portion, facing away from the source 1024, of the second drive signal line 1032, and an end portion, facing away from the drain 1025 of the third drive signal line 1033 is covered by the cover plate 200. Illustratively, an end, facing away from the detection unit 102, of the first drive signal line 1031 is provided with a first signal terminal G, an end, facing away from the detection unit 102, of the second drive signal line 1032 is provided with a second signal terminal S, and an end, facing away from the detection unit 102, of the third drive signal line 1033 is provided with a third signal terminal D. The first signal terminal G, the second signal terminal S, and the third signal terminal D are not covered by the cover plate 200. That is, orthogonal projections of the first signal terminal G, the second signal terminal S, and the third signal terminal D on the base substrate 101 are not overlapped with the orthogonal projection of the cover plate 200 on the base substrate 101. In this case, the drive assembly respectively applies corresponding electric signals to the first drive signal line 1031, the second drive signal line 1032, and the third drive signal line 1033 through the first signal terminal G, the second signal terminal S, and the third signal terminal D upon being bonded on the detecting back plate 100. Illustratively, in the case that the liquid under detection in the detection cavity V requires to be detected, the drive assembly applies a gate signal to the first drive signal line 1031 through the first signal terminal G, such that the gate 1023 is loaded with the gate signal, and the channel region in the graphene layer 1022 is conducted. Then, the drive assembly applies a detection signal to the second drive signal line 1032 through the second signal terminal S, such that the detection signal is transmitted to the third drive signal line 1033 through the source 1024, the channel region in the graphene layer 1022, and the drain 1025. As such, the drive assembly receives the detection signal transmitted on the third drive signal line 1033 through the third signal terminal D and detects the current of the detection signal to determine the magnitude of the current between the source 1024 and the drain 1025.

It should be noted that the insulative layer 1026 in the detection unit 102 wraps a portion of the first drive signal line 1031 in the detection cavity V and does not wrap the gate 1023. In some embodiments, the insulative layer 1026 in the detection unit 102 extends outwards the detection cavity V to wrap signal lines (for example, the first drive signal line 1031, the second drive signal line 1032, and the third drive signal line 1033) in the detection cavity V, such that the signal lines in the detecting back plate 100 are protected by the insulative layer 1026.

In the embodiments of the present disclosure, a shape of the orthogonal projection of the gate 1023 in the detection unit 102 on the base substrate 101 is diversified. Illustratively, FIG. 12 is illustrated by taking the shape of the orthogonal projection of the gate 1023 in the detection unit 102 on the base substrate 101 being crescent as an example. In some embodiments, the orthogonal projection of the gate 1023 in the detection unit 102 on the base substrate 101 is in a rectangular or circular shape, and the like, which is not limited in the embodiments of the present disclosure.

In some embodiments, a material of the insulative layer 1026 in the detection unit 102 is an organic photosensitive material. For example, the insulative layer 1026 is composed of the epoxy resin, the photosensitizer, and the organic solvent. In this case, a patterning process on a thin film is finished only by exposing and developing the thin film made of the photosensitive material in manufacturing the insulative layer 1026, such that the thin film is processed to the patterned insulative layer 1026. In some embodiments, the insulative layer 1026 in the detection unit 102 is made of an inorganic material, for example, silicon oxide, silicon nitride, and the like.

In the embodiments of the present disclosure, prior to detection on the liquid under detection by the biosensor, a plurality of detecting curves in one-to-one correspondence to the plurality of detection units are acquired, such that concentrations of various antigenic proteins in the liquid under detection are determined based on the plurality of detecting curves.

Illustratively, as the detection unit 102 in each detection cavity V in the biosensor 000 detects a concentration of one type of antigenic protein, a plurality of liquids mixed with one antigenic protein are prepared and injected to the biosensor 000, and the corresponding detecting curves are acquired by detecting the current between the source 1024 and the drain 1025. It should be noted that in the case that a liquid mixed with one antigenic protein is injected to the biosensor 000, a current of the detection unit 102 containing the antibody protein corresponding to the antigenic protein changes, and currents of other detection unit 102 do not change. Thus, the plurality of liquids mixed with the one antigenic protein are sequentially injected to the biosensor 000, and signals are acquired in several times to acquire the plurality of detecting curves. The following embodiments are described by taking the antigenic protein being Aβ40 and acquisition of detecting curves corresponding to the detection unit 102 under detection Aβ40 as an example.

Firstly, a buffer solution (for example, a phosphate buffered saline solution) not mixed with the antigenic protein is injected to the biosensor 000, and a current of a signal output by the drain 1025 in the detection unit 102 is determined and denoted as a first current I₀. Then, Aβ40 is mixed in the buffer solution, and a quantity of Aβ40 mixed in the buffer solution is increasingly increased over time, such that a concentration of Aβ 40 mixed in the buffer solution is increasingly increased. In addition, the current of the signal output by the drain 1025 in the detection unit 102 are determined all the time in the process, and the current is determined as a second current I₁. As reaction degrees of A β 40 of different concentrations with the corresponding antibody proteins in the detection unit 102 are different, the second current I₁ output by the drain 1025 in the detection unit 102 is continually changed with the increasing increment of the concentration of Aβ40.

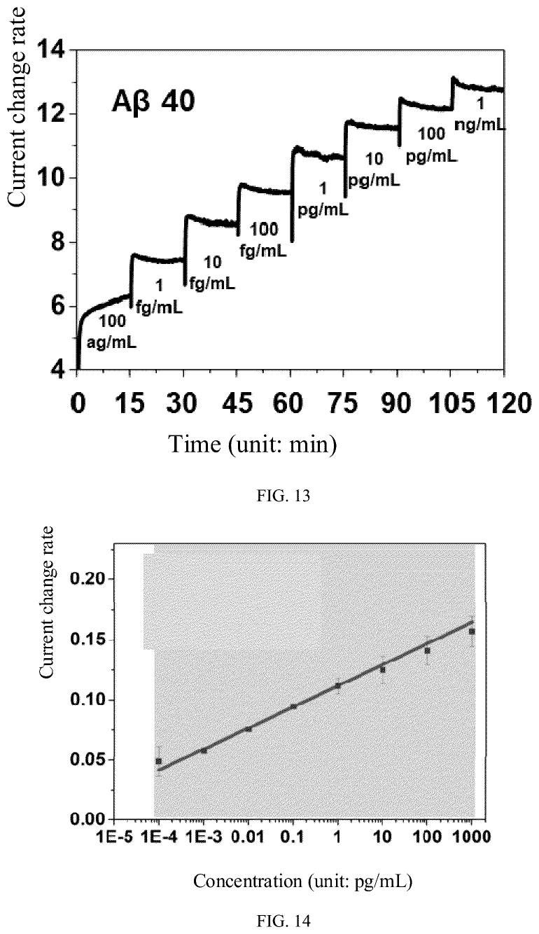

Referring to FIG. 13, FIG. 13 is a schematic diagram of a relationship of current change rates at different concentrations of Aβ40 according to some embodiments of the present disclosure. In FIG. 13, abscissae represent time, and ordinates represents a current change rate, that is, a ration of a difference between the second current I₁ and the first current I₀ to the first current I₀. The quantity of Aβ40 mixed in the buffer solution is increasingly increased over time, such that the concentration of Aβ40 mixed in the buffer solution is increasingly increased, and thus the current change rate is changed.

It should be noted that upon acquisition of the relationship of the current change rates at different concentrations of Aβ40 shown in FIG. 13, a plurality of same experiments require to be conducted to acquire a plurality of diagrams of the relationship shown in FIG. 13, and the plurality of diagrams of the relationship are fitted to acquire corresponding detecting curves. Referring to FIG. 14, FIG. 14 is a detecting curve of detecting Aβ40 by a detection unit according to some embodiments of the present disclosure. In FIG. 14, abscissae represent the concentration, and ordinates represents the current change rate. Upon acquisition of the detecting curve, in the case that the concentration of Aβ40 in the liquid under detection requires to be detected, the current change rate is first determined based on the current of the electric signal output by the drain 1025 in the detection unit 102, and the concentration corresponding to the current change rate is then determined based on the detecting curve. The concentration is the concentration of Aβ40 in the liquid under detection.

It should be noted that the detecting curve for determining the concentration of the antigenic protein in the liquid under detection is approximately linear, and thus the effect of detecting the concentration of the antigenic protein is great.

In summary, the biosensor in the embodiments of the present disclosure includes a detecting back plate and a cover plate. The detecting back plate includes a base substrate, a plurality of detection units on a side of the base substrate, and a first drive signal line electrically connected to a gate in the detection unit. After the cover plate is secured on the base substrate in the detecting back plate, each recess in the cover plate and the detecting back plate form a detection cavity, and the detection unit is in the detection cavity. As such, after a liquid under detection is accommodated in the detection cavity, and a drive signal is applied to the detection unit through a drive signal line, concentrations of biomolecules in the liquid under detection are detected by the detection unit. As the first drive signal line electrically connected to the gate in the detection unit is integrated in the detecting back plate, the detecting back plate directly applies a gate signal to the detection unit through the first drive signal line, without the need to holes in the cover plate for inserting metal wires to the detection cavity, such that the sealing between the cover plate and the detecting back plate is great, volatility of the liquid under detection in the detection cavity is efficiently reduced, and an accuracy of detecting the concentrations of the biomolecules in the liquid under detection by the detection unit is great.

Some embodiments of the present disclosure further provide a biochip. The biochip includes a drive assembly and a biosensor electrically connected to the drive assembly. The biosensor is the biosensor in the above embodiments, for example, the biosensor shown in FIG. 1 or FIG. 6. The drive assembly is electrically connected to an end portion, facing away from the detection unit 101, of the drive signal line 103 in the biosensor, such that the drive assembly transmits the drive signal to the detection unit 102 through the drive signal line 103 and receives the detection signal from the detection unit 102, such that the concentrations of the biomolecules in the liquid under detection are detected.

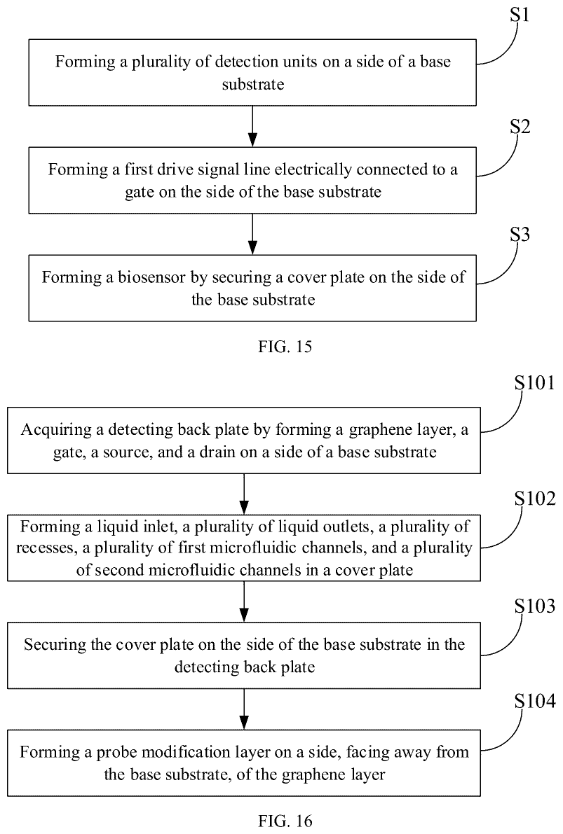

Some embodiments of the present disclosure further provide a method for manufacturing a biosensor. As shown in FIG. 15, FIG. 15 is a flowchart of a method for manufacturing a biosensor according to some embodiments of the present disclosure. The method for manufacturing the biosensor is applicable to manufacturing the biosensor shown in FIG. 1 or FIG. 6 and includes the following processes.

In S1, a plurality of detection units are formed on a side of the base substrate, wherein each of the plurality of detection units includes a gate.

In S2, a first drive signal line electrically connected to the gate is formed on the side of the base substrate.

In S3, a biosensor is acquired by securing a cover plate on the side of the base substrate.

A plurality of recesses configured to accommodate the plurality of detection units are defined in a side, proximal to the base substrate, of the cover plate.

In summary, in the method for manufacturing the biosensor, After the cover plate is secured on the base substrate in the detecting back plate, each recess in the cover plate and the detecting back plate form a detection cavity, and the detection unit is in the detection cavity. As such, after a liquid under detection is accommodated in the detection cavity, and a drive signal is applied to the detection unit through a drive signal line, concentrations of biomolecules in the liquid under detection are detected by the detection unit. As the first drive signal line electrically connected to the gate in the detection unit is integrated in the detecting back plate, the detecting back plate directly applies a gate signal to the detection unit through the first drive signal line, without the need to holes in the cover plate for inserting metal wires to the detection cavity, such that the sealing between the cover plate and the detecting back plate is great, volatility of the liquid under detection in the detection cavity is efficiently reduced, and an accuracy of detecting the concentrations of the biomolecules in the liquid under detection by the detection unit is great.

As shown in FIG. 16, FIG. 16 is a flowchart of another method for manufacturing a biosensor according to some embodiments of the present disclosure. The method for manufacturing the biosensor is applicable to manufacturing the biosensor shown in FIG. 6 and includes the following processes.

In S101, a detecting back plate is acquired by forming a graphene layer, a gate, a source, and a drain on a side of a base substrate.

In some embodiments, the base substrate is a silicon-based substrate or a glass substrate.

Illustratively, a graphene semiconductor thin film is formed on the base substrate by any one of depositing, coating, sputtering, and the like, and the graphene layer is formed by performing a one patterning process on the graphene semiconductor thin film.

Then, a metal thin film is formed in the side, facing away from the base substrate, of the graphene layer by any one of depositing, coating, sputtering, and the like, and a metal pattern is form by one patterning process on the metal thin film. The metal pattern includes the gate, the source, and the drain in the detection unit, and the drive signal line electrically connected to the detection unit. The orthogonal projection of the gate on the base substrate is not overlapped with an orthogonal projection of the graphene layer on the base substrate, and the source and the drain are lapped with the graphene layer.

In some embodiments, upon formation of the metal pattern on the side, facing away from the base substrate, of the graphene layer, an insulative thin film is formed by any one of depositing, coating, sputtering, and the like, and the insulative layer is form by one patterning process on the insulative thin film. The orthogonal projection of the gate on the base substrate is not overlapped with an orthogonal projection of the insulative layer on the base substrate, and the insulative layer wraps the source and the drain.

In S102, a liquid inlet, a plurality of liquid outlets, a plurality of recesses, a plurality of first microfluidic channels, and a plurality of second microfluidic channels are formed in the cover plate.

In some embodiments, the cover plate is made of PDMS.

Illustratively, a cover plater including a plurality of first microfluidic channels and a plurality of second microfluidic channels are manufactured by a molding process, a liquid inlet and a plurality of liquid outlets are disposed on one side of the cover plate, and a plurality of recesses are defined in the other side of the cover plate.

The plurality of first microfluidic channels are in one-to-one correspondence to the plurality of recesses. First ends of the plurality of first microfluidic channels are connected to the liquid inlet, and second ends of the plurality of first microfluidic channels are connected to the corresponding recesses. The plurality of second microfluidic channels are in one-to-one correspondence to the plurality of recesses and the plurality of liquid outlets. First ends of the plurality of second microfluidic channels are connected to the corresponding recesses, and second ends of the plurality of second microfluidic channels are connected to the corresponding liquid outlets.

In S103, the cover plate is secured on the side of the base substrate in the detecting back plate.

Illustratively, plasma bombardment is performed on a side of the cover plate with the recess before the cover plate is covered on the detecting back plate, such that the cover plate is closely attached to the detecting back plate upon being covered on the detecting back plate.

In S104, a probe modification layer is formed on a side, facing away from the base substrate, of the graphene layer.

Illustratively, a binding liquid is injected to the recesses through the liquid inlet. In some embodiments, the binding liquid is a DMSO solution of 1-Pyrenebutyric acid succinimidyl ester. The binding liquid is incubated for 3 h upon being injected to the recesses, and a solution containing different types of antibody proteins is sequentially injected to the plurality of recesses through the plurality of liquid outlets upon cleaning, such that the antibody proteins are bound with the binding substance. As such, the probe modification layer is formed on the side, facing away from the base substrate, of the graphene layer, an orthogonal projection of the probe modification layer on the base substrate is within the orthogonal projection of the graphene layer on the base substrate and between an orthogonal projection of the source on the base substrate and an orthogonal projection of the drain on the base substrate, and the probe modification layer contains an antibody protein for reacting with a specific antigenic protein in a liquid under detection. Eventually, the plurality of recesses are cleaned, and the probe modification layer is enclosed by 2-Aminoethanol or BSA.

In summary, in the method for manufacturing the biosensor, After the cover plate is secured on the base substrate in the detecting back plate, each recess in the cover plate and the detecting back plate form a detection cavity, and the detection unit is in the detection cavity. As such, after a liquid under detection is accommodated in the detection cavity, and a drive signal is applied to the detection unit through a drive signal line, concentrations of biomolecules in the liquid under detection are detected by the detection unit. As the first drive signal line electrically connected to the gate in the detection unit is integrated in the detecting back plate, the detecting back plate directly applies a gate signal to the detection unit through the first drive signal line, without the need to holes in the cover plate for inserting metal wires to the detection cavity, such that the sealing between the cover plate and the detecting back plate is great, volatility of the liquid under detection in the detection cavity is efficiently reduced, and an accuracy of detecting the concentrations of the biomolecules in the liquid under detection by the detection unit is great.

It should be noted that in the accompanying drawings, for clarity of the illustration, the dimension of the layers and regions may be scaled up. It should be understood that when an element or layer is described as being “on” another element or layer, the described element or layer may be directly located on other elements or layers, or an intermediate layer may exist. In addition, it should be understood that when an element or layer is described as being “under” another element or layer, the described element or layer may be directly located under other elements, or more than one intermediate layer or element may exist. In addition, it should be further understood that when a layer or element is described as being arranged “between” two layers or elements, the described layer or element may be the only layer between the two layers or elements, or more than one intermediate layer or element may exist. In the whole disclosure, like reference numerals indicate like elements.

In the present disclosure, the terms “first” and “second” are only used for the purpose of description and should not be construed as indicating or implying relative importance or implicitly indicating the number of technical features as indicated. Unless otherwise clearly defined, the expression “a plurality of” refers to two or more.

Described above are example embodiments of the present disclosure, and are not intended to limit the present disclosure. Any modifications, equivalent replacements, improvements and the like made within the spirit and principles of the present disclosure should be included within the scope of protection of the present disclosure.

1. A biosensor, comprising:
a base substrate;
a plurality of detection units disposed on a side of the base substrate, wherein each of the plurality of detection units comprises a gate;
a first drive signal line disposed on the side of the base substrate and electrically connected to the gate; and
a cover plate fixedly connected to the side of the base substrate, wherein a plurality of recesses are defined in a side, proximal to the base substrate, of the cover plate.

2. The biosensor according to claim 1, wherein each of the plurality of detection units further comprises a graphene layer and a probe modification layer on a side, facing away from the base substrate, of the graphene layer, wherein an orthogonal projection of the graphene layer on the base substrate is not overlapped with an orthogonal projection of the gate on the base substrate, an orthogonal projection of the probe modification layer on the base substrate is within the orthogonal projection of the graphene layer on the base substrate, and the probe modification layer contains an antibody protein for reacting with a specific antigenic protein in a liquid under detection.

3. The biosensor according to claim 2, wherein the probe modification layers in the plurality of detection units contain different types of antibody proteins.

4. The biosensor according to claim 2, wherein each of the plurality of detection units further comprises a source and a drain that are lapped with the graphene layer, wherein the orthogonal projection of the probe modification layer on the base substrate is between an orthogonal projection of the source on the base substrate and an orthogonal projection of the drain on the base substrate.

5. The biosensor according to claim 4, wherein the gate, the source, and the drain are disposed on a same layer and made of a same material.

6. The biosensor according to claim 4, wherein each of the plurality of detection units further comprises an insulative layer on a side, facing away from the base substrate, of the source and the drain, wherein the insulative layer is configured to wrap the source and the drain, wherein an orthogonal projection of the insulative layer on the base substrate is not overlapped with the orthogonal projection of the gate on the base substrate and the orthogonal projection of the probe modification layer on the base substrate.

7. The biosensor according to claim 6, further comprising: a second drive signal line and a third drive signal line that are disposed on the side of the base substrate, wherein the second drive signal line is electrically connected to the source, the third drive signal line is electrically connected to the drain, none of an end portion, facing away from the gate, of the first drive signal line, an end portion, facing away from the source, of the second drive signal line, and an end portion, facing way from the drain, of the third drive signal line is covered by the cover plate, and the insulative layer is further configured to wrap portions of the second drive signal line and the third drive signal line in the plurality of recesses.

8. The biosensor according to claim 1, wherein a liquid inlet and a plurality of liquid outlets are defined in a side, facing away from the base substrate, of the cover plate, wherein the liquid inlet is connected to the plurality of recesses, the plurality of liquid outlets are in one-to-one correspondence to the plurality of recesses and are connected to corresponding recesses, and orthogonal projections of the plurality of recesses on the base substrate are not overlapped with an orthogonal projection of the liquid inlet on the base substrate and orthogonal projections of the plurality of liquid outlets on the base substrate.

9. The biosensor according to claim 8, wherein the cover plate further comprises a plurality of first microfluidic channels in one-to-one correspondence to the plurality of recesses, wherein first ends of the plurality of first microfluidic channels are connected to the liquid inlet, and second ends of the plurality of first microfluidic channels are connected to corresponding recesses.

10. The biosensor according to claim 9, wherein each of the plurality of first microfluidic channels comprises a plurality of lengthening sub-channels and a plurality of connecting sub-channels, wherein the plurality of lengthening sub-channels and the plurality of connecting sub-channels are alternately arranged and are sequentially connected.

11. The biosensor according to claim 8, wherein the cover plate further comprises a plurality of second microfluidic channels in one-to-one correspondence to the plurality of recesses, wherein first ends of the plurality of second microfluidic channels are connected to corresponding recesses, and second ends of the plurality of second microfluidic channels are connected to corresponding liquid outlets.

12. A biochip, comprising: a drive assembly and a biosensor electrically connected to the drive assembly, wherein the biosensor comprises:
a base substrate;
a plurality of detection units disposed on a side of the base substrate, wherein each of the plurality of detection units comprises a gate;
a first drive signal line disposed on the side of the base substrate and electrically connected to the gate; and
a cover plate fixedly connected to the side of the base substrate, wherein a plurality of recesses are defined in a side, proximal to the base substrate, of the cover plate.

13. A method for manufacturing a biosensor, comprising:
forming a plurality of detection units on a side of a base substrate, wherein each of the plurality of detection units comprises a gate;
forming a first drive signal line electrically connected to the gate on the side of the base substrate; and
securing a cover plate on the side of the base substrate, wherein a plurality of recesses configured to accommodate the plurality of detection units are defined in a side, proximal to the base substrate, of the cover plate.

14. The method according to claim 13, wherein forming the plurality of detection units on the side of the base substrate comprises:
forming a graphene layer, a gate, a source, and a drain on the side of the base substrate, wherein the source and the drain are both lapped with the graphene layer, and an orthogonal projection of the gate on the base substrate is not overlapped with an orthogonal projection of the graphene layer on the base substrate; and
forming a probe modification layer on a side, facing away from the base substrate, of the graphene layer upon securing the cover plate on the side of the base substrate, wherein an orthogonal projection of the probe modification layer on the base substrate is within the orthogonal projection of the graphene layer on the base substrate and between an orthogonal projection of the source on the base substrate and an orthogonal projection of the drain on the base substrate, and the probe modification layer contains an antibody protein for reacting with a specific antigenic protein in a liquid under detection.

15. The method according to claim 14, wherein
a liquid inlet and a plurality of liquid outlets are defined in a side, facing away from the base substrate, of the cover plate, wherein the liquid inlet is connected to the plurality of recesses, and the plurality of liquid outlets are in one-to-one correspondence to the plurality of recesses and are connected to corresponding recesses; and
forming the probe modification layer on the side, facing away from the base substrate, of the graphene layer comprises:
forming a binding substance on the graphene layer by injecting a binding solution to the plurality of recesses through the liquid inlet; and
sequentially injecting a solution containing different types of antibody proteins to the plurality of recesses through the plurality of liquid outlets, such that the antibody proteins are bound to the binding substance.

16. The biochip according to claim 12, wherein each of the plurality of detection units further comprises a graphene layer and a probe modification layer on a side, facing away from the base substrate, of the graphene layer, wherein an orthogonal projection of the graphene layer on the base substrate is not overlapped with an orthogonal projection of the gate on the base substrate, an orthogonal projection of the probe modification layer on the base substrate is within the orthogonal projection of the graphene layer on the base substrate, and the probe modification layer contains an antibody protein for reacting with a specific antigenic protein in a liquid under detection.

17. The biochip according to claim 16, wherein the probe modification layers in the plurality of detection units contain different types of antibody proteins.

18. The biochip according to claim 16, wherein each of the plurality of detection units further comprises a source and a drain that are lapped with the graphene layer, wherein the orthogonal projection of the probe modification layer on the base substrate is between an orthogonal projection of the source on the base substrate and an orthogonal projection of the drain on the base substrate.

19. The biochip according to claim 18, wherein the gate, the source, and the drain are disposed on a same layer and made of a same material.

20. The biochip according to claim 18, wherein each of the plurality of detection units further comprises an insulative layer on a side, facing away from the base substrate, of the source and the drain, wherein the insulative layer is configured to wrap the source and the drain, wherein an orthogonal projection of the insulative layer on the base substrate is not overlapped with the orthogonal projection of the gate on the base substrate and the orthogonal projection of the probe modification layer on the base substrate.
